(19) United States
(12) Patent Application Publication
(10) Pub. No.: US 2009/0130235 A1
(43) Pub. Date: May 21, 2009

(54) METHOD FOR MARKETING VIBRATION MEDICINE PRODUCTS

(76) Inventor: Ilan Azouri, Tel Aviv (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(21) Appl. No.: 12/269,902

(22) Filed: Nov. 13, 2008

Related U.S. Application Data
(60) Provisional application No. 60/989,123, filed on Nov. 20, 2007.

(51) Int. Cl. A61K 36/00; G06Q 90/00; A61P 25/00

(52) U.S. Cl. 424/725; 705/500

(57) ABSTRACT

It is disclosed a method for marketing vibration medicine products. The method includes getting an original liquid vibration medicine product, producing a powder vibration medicine product, providing that powder vibration medicine product for blending inside a food or drink entity to get a new entity, and marketing the new entity as an improved first entity. The original vibration medicine product includes essences, and is marketed as having properties associated with the essences. The production of the second product is done using the original vibration medicine product and an acceptable food carrier as raw materials. Consequently, the second product has some presence of the original essences. The second entity is improved over the first entity in its association with the first vibration medicine product.

### CROSS-REFERENCE TO RELATED APPLICATIONS

This application claims the benefits of U.S. provisional patent application Ser. No. 60/989,123 filed Nov. 20, 2007 by the present inventor.

### BACKGROUND OF THE INVENTION

1. Field of the Invention

The invention is in the field of marketing products of alternative or complementary medicine. In particular, the invention deals with new methods to market vibration medicine products to the mass market.

2. Description of Related Art

Vibration Medicine (hereafter VM) is considered by its practitioners to be associated with “energy”, “frequency” and “vibrations” rather than the chemical and biological content of conventional medicines that work on the bio-physical dysfunctions. Vibration medicine is mainly used in complementary medicine and holistic fields such as homeopathy, vibration based devices (Hado) as developed by Massaru Emoto of Japan, B-com and Quaxi devices, etc. The term “vibration medicine” as used herein refers mainly to natural vibration medicine that uses natural elements like flower extractions and flower essences as in Bach flowers.

Bach Flower Remedies (also known as flower essences) have been developed since 1936 by Eduard Bach and followers, who have attributed the remedies unique balancing effect on user well being. Currently, there are over hundred brands worldwide, which offer natural sources for their vibration products using flowers, animals, human cells, sea essences, natural sacred places and phenomena like “vibrations” of moon light or a storm.

Vibration Medicine can be used as a balancing support to many people in dealing with a vast variety of physical and psychological situations, symptoms and imbalances (e.g., stress), as well as supporting personal development. Moreover, VM is considered absolutely safe with no restrictions whatsoever.

Most of the VM medicines are delivered in water-alcohol solutions (approximately 25%), wherein water is used as a carrier and alcohol is used as a preservatives. Flower essences are distributed mainly by practitioners and natural health shops and pharmacies in small glass bottles (typically 30 ml bottles) and in droppers with which the customer instill drops into his mouth. In homeopathy, the most recognized VM field, the common application method is by small pellets or globules which melt in consumer mouth.

The principles of VM are hard to capture and rather “intangible”. Therefore, VM is presently a relatively small market with low consumption and usage. Development of new Flower Remedy products led to a small increase of the market, but yet there is no access to the mass market. It seems that a bottleneck exists at the consumer side, preventing large scale market distribution, a bottleneck due to limited awareness and understanding of VM products. Thus, the mass market access necessitates special delivery and communication approach and new distribution channels.

One objective of the present invention is to bring natural Vibration Medicine to the mass market, and thus to increase worldwide vibration medicine consumption, and provide commercial success to active companies.

The inventor laid down the challenge to create and deliver new daily basic products—Vibration or “flower Essence”—products, that can support better balance, personal development and increased conscious awareness. The inventor asserts that should people in global mass market have friendly and easy access for using these products, they may maintain better balance and awareness, and it may help in creating a positive effect on future of humankind.

### BRIEF DESCRIPTION OF THE DISCLOSURE

It is disclosed for the first time a method for marketing vibration medicine products. The method includes getting an original first vibration medicine product of a certain physical shape, producing another vibration medicine product at a different physical shape, providing that second vibration medicine product for blending inside a first entity to get a second entity, and marketing the second entity as an improved first entity. The original vibration medicine product includes essences, and is marketed as having properties associated with the essences. The production of the second product is done using the original vibration medicine product and an acceptable food carrier as raw materials. Consequently, the second product has some presence of the original essences. The first entity and the second entity are either drink and food. The second entity is improved over the first entity in its association with the first vibration medicine product.

In some embodiments, the original vibration medicine product is a brand vibration medicine product. It may include water and alcohol at 20% to 60% alcohol rating, preferably 25% alcohol rating.

Exemplary vibration medicine product are Bach flowers remedies, Australian Bush flowers, Alaskan Flower essences and Pacific essences.

Exemplary essences are essences of the following plants: agrimoni, aspen, beech, centaury, cerato, cherry plum, chestnut bud, chicory, clematis, crab apple, elm, gentian, gorse, heather, holly, honeysuckle, hornbeam, impatiens, larch, mimulus, mustard, oak, olive, pine, red chestnut, rock rose, rock water, scleranthus, star of bethlehem, sweet chestnut, vervain, vine, walnut, water violet, water chestnut, wild oat, wild rose, and willow.

Exemplary properties associated with essences are balancing undesirable characteristics, balancing undesirable emotional states, supporting personal development and transformation of state of mind.

Exemplary acceptable food carriers are sucrose, glucose, lactose and xylitol.

In some embodiments, the original product is liquid and the result product is a powder. Exemplary methods for production of the second vibration medicine product are:
(A) Spraying several drops of the first product on sucrose globules while mixing, allowing the mixture to dry and grinding the globules into a powder of a desired particle size.
(B) Spraying several drops of the first product on sugar crystals while mixing, allowing the mixture to dry, testing for dissolution, and if solubility is poor, keeping on grinding up to getting particles at a desired size.
(C) Spraying several drops of the first product on icing sugar, while mixing, and allowing the mixture to dry.

In some embodiments, the second product is provided to end users, wherein an end user may blend the second product in a drink serving or in a food serving, getting a mixture which may be consumed while it is still fresh.

In some embodiments, one provides the second vibration medicine product as packed personal doses, each with a personal serving of the first entity. An end user may get the combined product, blend the personal dose into the personal serving and consume the resulted second entity mixture while it is still fresh. For example, the personal serving of the first entity is a bottle of mineral water and the VM personal dose is packed within the bottle cork assembly.

In some embodiments, the second product is sold to a manufacturer of the first entity. The manufacturer adds the second product to the first entity as a supplement to get the second entity. Consequently, the manufacturer provides the second entity to end users using the very marketing channels used for marketing of the first entity and, if needed, marketing channels especially developed for the second entity.

The improvement of the second entity over the first entity may be that it has a property associated with the original essences. In case that the first vibration medicine product is a brand vibration medicine product, the improvement may be association of the second entity to the brand name of the vibration medicine product.

Exemplary first entities are mineral water and soft drinks.

It is disclosed for the first time, a method for vibration medicine products. The method includes getting a brand vibration medicine product, providing the brand vibration medicine product for blending inside a first entity to get a second entity and marketing the second entity as an improved first entity. The improvement is associated with the brand vibration medicine product.

In some embodiments, the brand vibration medicine is provided to a manufacturer of the first entity.

Exemplary brand vibration medicine product are brand Bach flowers remedies, brand Australian Bush flowers, brand Alaskan Flower essences and brand Pacific essences.

It is disclosed for the first time a method for marketing vibration medicine products. The method includes getting a brand vibration medicine product, and packing it into personal doses. An end user may take a packed personal dose, blend it with a personal serving of food or drink and may consume the mixture while it is still fresh.

These and further embodiments will be apparent from the detailed description and examples that follow.

### BRIEF DESCRIPTION OF THE DRAWINGS

The subject matter regarded as the invention is particularly pointed out and distinctly claimed in the concluding portion of the specification. The invention, however, both as to organization and method of operation, together with features and advantages thereof, may best be understood by reference to the following detailed description when read with the accompanied drawings in which:

FIG. 1 is a flow chart of a first method for marketing vibration medicine products.

FIG. 2 shows two tasks which may be included in the step of providing the second vibration medicine product.

FIG. 3 is a flow chart of a second method for marketing vibration medicine product according to some embodiments.

FIG. 4 is a flow chart of a third method for marketing vibration medicine product according to some embodiments.

### DETAILED DESCRIPTION OF THE INVENTION

The present invention will now be described in terms of specific example embodiments. It is to be understood that the invention is not limited to the example embodiments disclosed. It should also be understood that not every feature of the methods for marketing VM products is necessary to implement the invention as claimed in any particular one of the appended claims. It should also be understood that throughout this disclosure, where a method is shown or described, the steps of the method may be performed in any order or simultaneously, unless it is clear from the context that one step depends on another being performed first.

Before explaining at least one embodiment of the invention in detail, it is to be understood that the invention is not limited in its application to the details set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Unless otherwise defined, all technical and scientific terms used herein have the same meaning as commonly understood by one of ordinary skill in the art to which this invention belongs. The materials, methods, and examples provided herein are illustrative only and not intended to be limiting.

In the description and claims of the present application, each of the verbs “comprise”, “include” and “have”, and conjugates thereof, are used to indicate that the object or objects of the verb are not necessarily a complete listing of members, components, elements or parts of the subject or subjects of the verb.

The marketing method of the present invention according to one embodiment is based on turning vibration medicine into a powder that can be added as a supplement to various products that may be consumed on a daily basis by people. Thus, one takes daily consumed products and enhance them with vibration medicine, enjoying a new category of products and supplements. By doing so, the vibration medicine is turned from a niche market product with limited consumption into a broadly consumed product.

The formulated powders can be used in various ways and in a vast range of products as foods and beverages, water systems and filters and containers, body paints and colors to be absorbed in the skin, cosmetics clothing, print paper based products as such as books or lottery tickets and fashion accessories. For example, in tattoo products, one may produce vibration medicine “tattoo” stickers by adding VM formulas to coloring and printing materials, and the vibration essence formula is absorbed into the skin once it is used in the conventional way. Note that a similar technique is used with Nicotine to help tobacco addicts overcome smoking termination. Similar stickers or patches may be attached to a bottled water label or a T-shirt, respectively, and add vibration based value and function to the product. Also, VM based stickers may be used within and/or attached and/or merged a label mechanism such as bottles shrink label, used for dairy product label, clothing or ally other type of goods.

The powder created can be mixed and add value to other products that have value in consumers perspectives (i.e. minerals, food supplements etc.), creating a combination with high value that will be more appealing to the consumer than each component alone.

The innovative marketing methods described here allow food and beverage producers to come up with new lines of products, adding value for their customers, and increasing their sales. In addition, the methods create new channels for the vibration medicine producers to sell their products increasing substantially their revenues and profits.

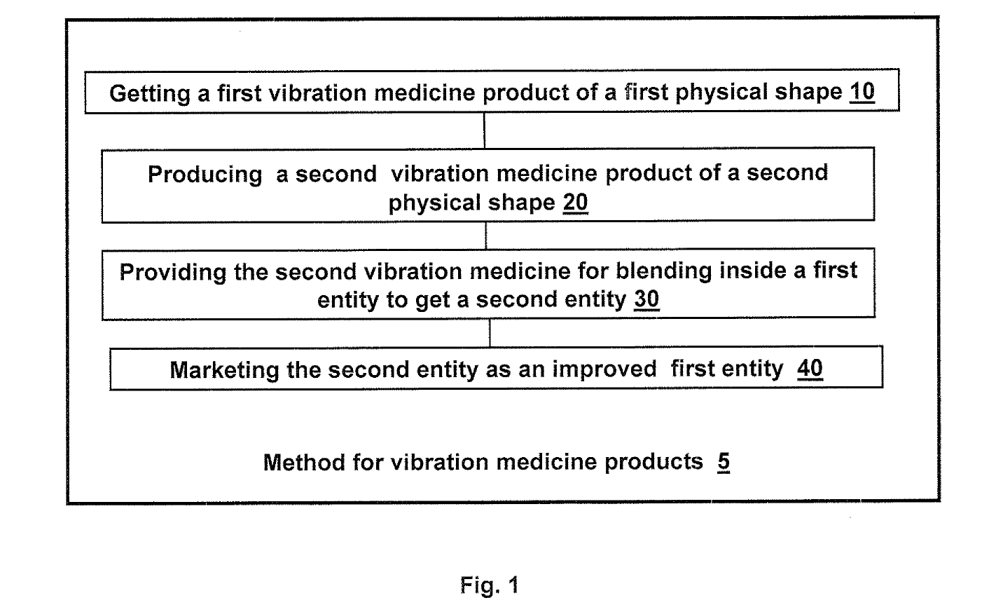

FIG. 1 shows a flowchart of a method 5 for marketing vibration medicine products. The method includes getting 10 an original first vibration medicine product of a certain physical shape, producing 20 another vibration medicine product at a different physical shape, providing 30 that second vibration medicine product for blending inside a first entity to get a second entity, and marketing 40 the second entity as an improved first entity. The original vibration medicine product includes essences, and is marketed as having properties associated with the essences. The production 20 of the second product is done using the original vibration medicine product and an acceptable food carrier as raw materials. Consequently, the second product has some presence of the original essences. The first entity and the second entity are either drink and food. The second entity is improved over the first entity in its association with the first vibration medicine product.

In some embodiments, the original vibration medicine product is a brand vibration medicine product. It may include water and alcohol at 20% to 60% alcohol rating, preferably 25% alcohol rating.

Exemplary vibration medicine products are Bach Flowers Remedies, Australian Bush flowers, Alaskan Flower essences and Pacific essences.

Exemplary essences are essences of the following plants: agrimoni, aspen, beech, centaury, cerato, cherry plum, chestnut bud, chicory, clematis, crab apple, elm, gentian, gorse, heather, holly, honeysuckle, hornbeam, impatiens, larch, mimulus, mustard, oak, olive, pine, red chestnut, rock rose, rock water, scleranthus, star of bethlehem, sweet chestnut, vervain, vine, walnut, water violet, water chestnut, wild oat, wild rose, and willow.

Exemplary first entity are mineral water and soft drinks.

Exemplary properties associated with essences are balancing undesirable characteristics, balancing undesirable emotional states, and supporting personal development.

Exemplary acceptable food carriers are sucrose, glucose, lactose and xylitol. Sucrose is a particularly acceptable carrier as it has several attractive features:

traditional use as homeopathic carrier, availability in different size grades, cheapness, having good crystal strength, having relatively large aqueous dissolution rate, being free of allergenic potential, having certain sweetness which may be required to add taste or mask other taste, having low moisture content, and no crystal aggregation to larger crystal in manufacture or storage.

The other sugars are less attractive. Glucose has a negative heat of solution which militates against ease of dissolution. Lactose has low water solubility and is not very sweet. In addition, due to lactose milky origin it may contain allergic impurities. Xylitol is expensive and is not readily available in the desired particle size.

Note that some packaging manufacturers require exact powder specifications, i.e. particles should be bigger than 30 microns in order to avoid loss of flowability.

Therefore, the water soluble carrier preferably should have a particle size of 50 to 100 microns. Preliminary experiments with sucrose globules produced slow dissolution rates after vigorous shaking.

In some embodiments, the original product is liquid and the result product is a powder. The powder is kept isolated from any liquid up until it is to be consumed. For example, when the user wants to consume the VM, she mixes the powder with water, allowing good VM absorption and activity.

Exemplary methods for production 20 of a powder vibration medicine product are:
(A) Spraying several drops of the first product on sucrose globules while mixing, allowing the mixture to dry and grinding the globules into a powder of a desired particle size. For example, one may spray the equivalent of 3-5 drops of VM brandy solution (25%) on 10 grams of sucrose globules while mixing, may allow the mixture to dry and then may grind the globules into a powder with the desired particle size (50-100 microns).
(B) Spraying several drops of the first product on sugar crystals while mixing, allowing the mixture to dry, testing for dissolution, and if solubility is poor, keeping oil grinding up to getting particles at a desired size. For example, one may spray the equivalent of 3-5 drops VM brandy solution on 10 grams of sucrose in the form of regular sugar crystals, may allow the mixture to dry and then may test it for dissolution. If solubility is poor he may keep on grinding into powder of smaller size particles depending on production requirements, i.e. not below 30 microns size limit. Note that brandy is used for preservation purposes and there are other possible preservatives such as vegetable glyceryne.
(C) Spraying several drops of the first product on icing sugar, while mixing, and allowing the mixture to dry.

The desirable size of both the sugar particles and the herbal essence should be approx. 50 microns (325 mesh) for fast dissolution or fine dispersion. Water content should be less than 2%.

In some embodiments, the second product is provided 30 to end users, wherein an end user may blend the second product a drink serving or a food serving, getting a mixture which may be consumed while it is still fresh.

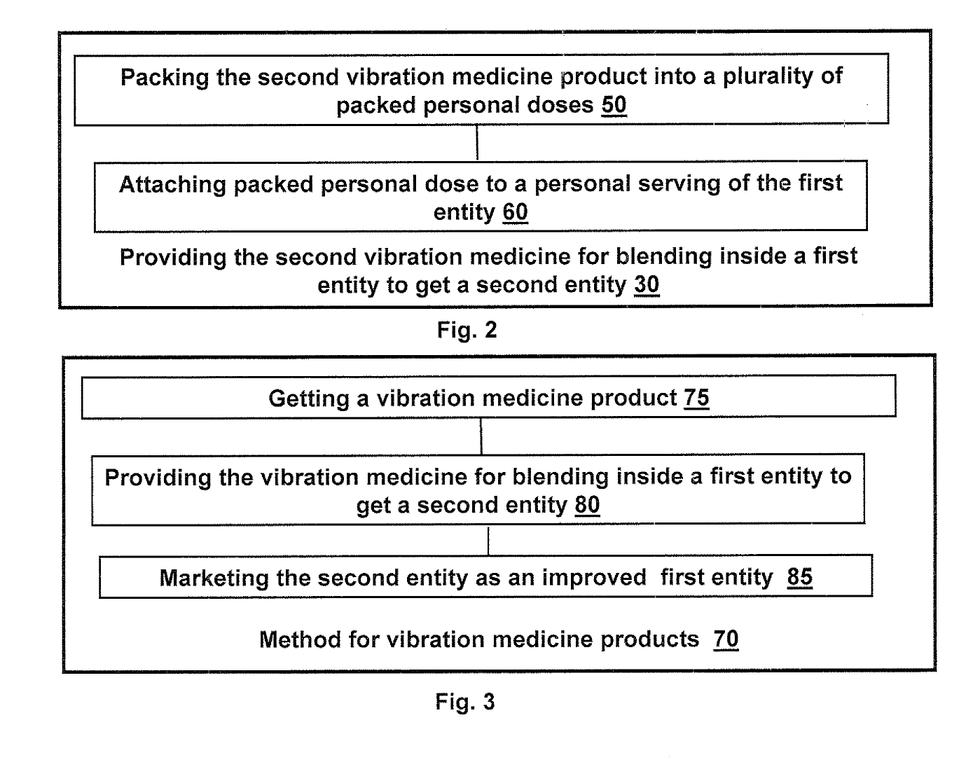

In some embodiments, described in the flowchart of FIG. 2, one provides 30 the second vibration medicine product as packed 50 personal doses with an attached 60 personal serving of the first entity. An end user may get the combined product, blend the personal dose into the personal serving and consume the mixture while it is still fresh. In one example, the personal serving of the first entity is a personal bottle of mineral water and the VM personal dose is packed within the bottle cork assembly. A special cork assembly, or cap, contains the powdered formula, isolating the powder from the liquid until usage. Thus the powder is kept stable for a longer period of time, and may be used in the moment of consumption without the need for any preservatives such as alcohol, or any expensive preserving technology.

Appropriate exemplary caps are Shupon cap, www.shupon-international.com, Vizdrink cap, www.vizdrink.com, vita-cap, blast max, POWERCAP.

Exemplary personal packaging for the personal VM doses are “tea bags” package type, and pearl shaped pill. In the case of “tea bags” package type, the blending with a drink serving means that the bag is inserted into a drink glass and the whole bag is shaken to stimulate powder mixing with the drink.

In some embodiments, the second product is sold to a manufacturer of the first entity. The manufacturer adds the second product to the first entity as a supplement to get the second entity. Consequently, the manufacturer provides the second entity to end users using the very marketing channels used for marketing of the first entity and, if needed, marketing channels especially developed for the second entity.

The improvement of the second entity over the first entity may be that it has a property associated with the original essences. In case that the first vibration medicine product is a brand vibration medicine product, an additional improvement may be association of the second entity to the brand name of the vibration medicine product.

In other preferred embodiment, the powder VM is blended into candies in their production process.

FIG. 3 depicts a flowchart of a method 70 for marketing vibration medicine products. The method includes getting 75 a brand vibration medicine product, providing 80 the brand vibration medicine product for blending inside a first entity to get a second entity, and marketing 85 the second entity as an improved first entity. The improvement is associated with the brand vibration medicine product. In one example the brand vibration medicine product is a liquid which is used as a liquid in blending inside the first entity.

In some embodiments, the brand VM product is blended into candies in their production process.

In some embodiments, the brand vibration medicine is provided to a manufacturer of the first entity. The manufacturer adds the brand vibration medicine to the first entity and sells it to end users as an improved first entity. For example, the manufacturer may be a mineral water supplier which gets a license from the brand company to use the brand name trademark in advertising improved mineral water bottles which contain some amount of the brand vibration medicine product. The advertisements indicate that the improved mineral water has properties attributed to the brand vibration medicine product. Thus, the prestige and fame of a well known product, sold in small quantities, are transferred to a new product, well fitted to marketing in the mass market.

Exemplary brand vibration medicine product are brand Bach flowers remedies, brand Australian Bush flowers, brand Alaskan Flower essences and brand Pacific essences.

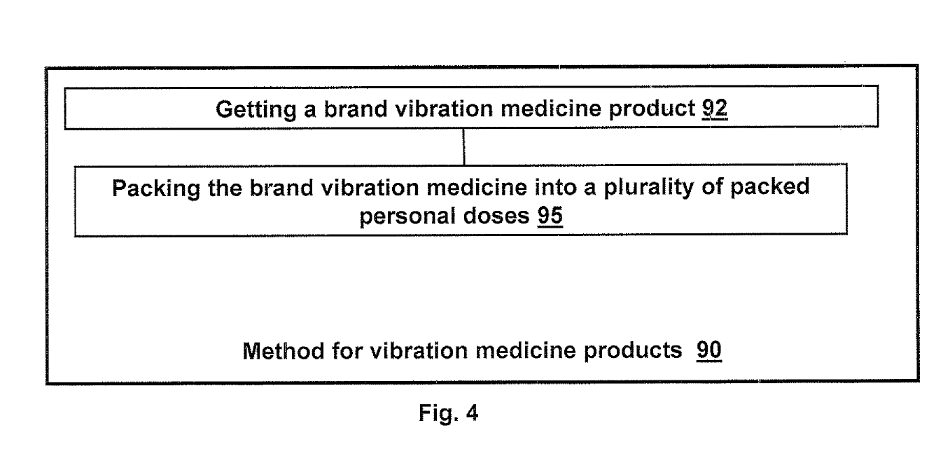

FIG. 4 shows a method 90 for marketing vibration medicine products. The method includes getting 92 a brand vibration medicine product, and packing it 95 into personal doses. An end user may take a packed personal dose, blend it with a personal serving of food or drink and may consume the mixture while it is still fresh.

The present invention is not limited to natural vibration medicine and includes also vibration medicine that is produced by artificial means such as by using music, machines such as B-com, Quantum Quaxi, etc, or any other method known in the art.

Although the invention has been described in conjunction with specific embodiments thereof it is evident that many alternatives, modifications and variations will be apparent to those skilled in the art. Accordingly, it is intended to embrace all such alternatives, modifications and variations that fall within the spirit and broad scope of the appended claims.

1. A method for vibration medicine products, the method including:
(a) getting a first vibration medicine product of a first physical shape, the product comprising:
(i) al least one essence; and
(ii) being marketed as having at least one property associated with said at least one essence;
(b) producing a second vibration medicine product of a second physical shape using two or more raw materials, said two or more raw materials including said first vibration medicine product and an acceptable food carrier, said second physical shape being different from said first physical shape;
(c) providing the second product for blending inside a first entity to get a second entity having some presence of said at least one essence, each of said first entity and said second entity being one of drink and food; and
(d) marketing said second entity as being an improvement of said first entity, the improvement being associated with the first vibration medicine product.

2. The method of claim 1 wherein said first vibration medicine product further comprises at least one of:
(iii) being a brand vibration medicine product; and
(iv) including water and alcohol at 20% to 60% alcohol rating.

3. The method of claim 1 wherein said second vibration medicine product is sold to end users.

4. The method of claim 1 wherein said first physical shape is a liquid.

5. The method of claim 1 wherein said first vibration medicine product is selected of the group consisting of Bach flowers remedies, Australian Bush flowers, Alaskan Flower essences and Pacific essences.

6. The method of claim 1 wherein said at least one essence is an essence of a plant selected from the group of plants consisting of agrimoni, aspen, beech, centaury, cerato, cherry plum, chestnut bud, chicory, clematis, crab apple, elm, gentian, gorse, heather, holly, honeysuckle, hornbeam, impatiens, larch, mimulus, mustard, oak, olive, pine, red chestnut, rock rose, rock water, scleranthus, star of bethlehem, sweet chestnut, vervain, vine, walnut, water violet, water chestnut, wild oat, wild rose, and willow.

7. The method of claim 1 wherein said at least one property includes balancing at least one of undesirable characteristic and undesirable emotional state.

8. The method of claim 1 wherein said at least one property includes one of supporting personal development and transformation of state of mind.

9. The method of claim 1 wherein said acceptable food carrier is one of sucrose, glucose, lactose and xylitol.

10. The method of claim 1 wherein said second physical shape is a powder.

11. The method of claim 10 wherein said producing a second vibration medicine product is done by one of:
(A) spraying several drops of the first product on sucrose globules while mixing, allowing the mixture to dry and grinding the globules into a powder of a desired particle size;
(B) spraying several drops of the first product on sugar crystals while mixing, allowing the mixture to dry, testing for dissolution, and if solubility is poor, keeping on grinding up to getting particles at a desired size; and
(C) spraying several drops of the first product on icing sugar, while mixing, and allowing the mixture to dry.

12. The method of claim 1 wherein said providing of the second product includes selling to end users, wherein an end user of said end users may blend the second product into one of a drink serving and a food serving to get a mixture and may consume the mixture while it is still fresh.

13. The method of claim 1 wherein said providing of the second product includes:
(A) packing the second product into a plurality of packed personal doses; and
(B) attaching each packed personal dose to a personal serving of said first entity; wherein an end user may get said personal serving together with the attached packed personal dose of said second product, and may blend that personal dose into said personal serving and may consume the resulted second entity mixture while it is still fresh.

14. The method of claim 13 wherein the personal doses are packed within cork assemblies of drink bottles.

15. The method of claim 1 wherein said providing of the second product includes selling of the second product to a manufacturer of said first entity, wherein said manufacturer may add the second product to said first entity as a supplement to get said second entity, and said manufacturer may provide said second entity to a plurality of end users using at least one of marketing channels available for marketing of said first entity and marketing channels especially developed for said second entity.

16. The method of claim 1 wherein the first entity improvement associated with the first vibration medicine product is having a property associated with said at least one essence.

17. The method of claim 1 wherein said first vibration medicine product is a brand vibration medicine product, and the improvement associated with the first product is association of the second entity to said brand vibration medicine product.

18. The method of claim 1 wherein said first entity is one of mineral water and a soft drink.

19. A method for vibration medicine products, the method including:
(a) getting a brand vibration medicine product, the product comprising:
(i) at least one essence; and
(ii) being marketed as having at least one property associated with said at least one essence;
(b) providing said brand vibration medicine product for blending inside a first entity to get a second entity having some presence of said at least one essence, each of said first entity and said second entity being one of drink and food; and
(c) marketing said second entity as being an improvement of said first entity, the improvement being associated with said brand vibration medicine product.

20. The method of claim 19 wherein said at least one essence is an essence of a plant selected from the group consisting of agrimoni, aspen, beech, centaury, cerato, cherry plum, chestnut bud, chicory, clematis, crab apple, elm, gentian, gorse, heather, holly, honeysuckle, hornbeam, impatiens, larch, mimulus, mustard, oak, olive, pine, red chestnut, rock rose, rock water, scleranthus, star of bethlehem, sweet chestnut, vervain, vine, walnut, water violet, water chestnut, wild oat, wild rose, and willow.

21. The method of claim 19 wherein said providing of said brand vibration medicine product is selling to a manufacturer of said first entity, wherein said manufacturer adds the first product to said first entity as a supplement to get said second entity, and said manufacturer provides said second entity to a plurality of end users using one of marketing channels available for marketing of said first entity and marketing channels especially developed for said second entity.

22. The method of claim 19 wherein said brand vibration medicine product further includes water and alcohol at 20% to 60% alcohol rating.

23. The method of claim 19 wherein said brand vibration medicine product is selected of the group consisting of brand Bach flowers remedies, brand Australian Bush flowers, brand Alaskan Flower essences and brand Pacific essences.

24. A method for vibration medicine products, the method including:
(a) getting a brand vibration medicine product, the product comprising:
(i) at least one essence; and
(ii) being marketed as having at least one property associated with said at least one essence;
and
(b) packing the brand vibration medicine product into a plurality of packed personal doses;
wherein an end user may take a packed personal dose, blend it with a personal serving of food or drink and may consume the mixture while it is still fresh.
